(19) United States
(12) Patent Application Publication
(10) Pub. No.: US 2023/0372146 A1
(43) Pub. Date: Nov. 23, 2023

(54) INTRAORAL SPEECH DEVICES, METHODS, AND SYSTEMS

(71) Applicant: The University of North Carolina at Chapel Hill, Chapel Hill, NC (US)

(72) Inventor: Rishma SHAH, Chapel Hill, NC (US)

(21) Appl. No.: 18/030,548

(22) PCT Filed: Oct. 8, 2021
(86) PCT No.: PCT/US2021/054107

§ 371 Date: Apr. 6, 2023

Related U.S. Application Data
(60) Provisional application No. 63/089,891, filed on Oct. 9, 2020.

(51) Int. Cl. A61F 5/58; G09B 19/04; A61B 5/00

(52) U.S. Cl. CPC A61F 5/58; G09B 19/04; A61B 5/682; A61B 5/4552; A61B 5/4803; A61B 2562/0247

(57) ABSTRACT

Intraoral speech devices, methods, systems and computer-implemented programs. An intraoral speech device or system can include a personalized intraoral speech device that detects and records tongue position and force, and speech sounds with correct articulation and provides the user with haptic, tactile, and/or audio-visual feedback on desired or correct tongue position. A mobile gaining-style application can be provided with the intraoral device that wirelessly receives the device signal with desired or correct tongue position providing physical or visual feedback.

### PRIORITY CLAIM

This patent application claims priority to U.S. Provisional Patent Ser. No. 63/089,891, filed Oct. 9, 2020, the disclosure of which is incorporated by reference herein in its entirety.

### TECHNICAL FIELD

The subject matter disclosed herein relates generally to speech devices and methods. More particularly, the subject matter disclosed herein relates to intraoral speech devices, methods, systems and computer-implemented programs and products.

### BACKGROUND

Speech impediments exist in greater than 8% of the population in the United States. Speech unintelligibility can have a negative impact on societal integration and psychosocial wellbeing of a person suffering from the pathology. Patients need regular, costly, speech therapy sessions to improve speech intelligibility. Thus, there is a need for a speech encouragement or correction device and/or system that can help quickly and easily improve the speech of those with speech disorders due to various causes. There is also a need to aid those who wish to affect their speech in a specific manner.

Recent studies have shown better compliance with mobile games compared to traditional methods of improving patient compliance. Thus, a system incorporating an intraoral speech device with a mobile compliance application would be an ideal solution to the speech impediments found in those with speech impediments.

### SUMMARY

In accordance with the subject matter here, intraoral speech devices, methods, systems and computer-implemented programs and products are disclosed. In some embodiments, an intraoral speech device or system can comprise a personalized intraoral speech device and mobile application that records tongue position and force with correct articulation and provides a user or patient with haptic, tactile, and/or audio-visual feedback, such as with tongue positioning, placement or pressure, and can, for example, comprise providing feedback based upon tongue position or placement relative to a desired or correct tongue position or placement associated with speech. In some embodiments, speech sounds may at the same time be detected in the mobile application. The approach involves customized construction of a variable thickness plate constructed of any suitable material, such as for example acrylic or polypropylene, with or without retentive clasps that cover the palate. A wireless-enabled (e.g., Bluetooth) touch-sensing surface or ultrasonic sensors can be included that can register a specific tongue position, placement, and force, confirm desired or correct articulation or provide information on incorrect tongue placement and/or force. A mobile gaming-style application can be provided for use in association with the intraoral device that receives the device signal with tongue positioning or placement, such as desired or correct tongue positioning or placement, to provide physical and/or audio-visual feedback. Haptic feedback can, for example, comprise plate vibration with specified tongue positioning, placement or pressure, and can, for example, comprise desired or correct tongue placement. Tactile feedback is with, for example different materials and surfaces. Haptic feedback, or any other feedback, may also occur from the mobile application to the device thus training/teaching the user or patient on the desired tongue placement and force. Speech sounds registered in the mobile application can also be displayed to provide immediate audio-visual feedback.

In one aspect, an intraoral speech device or system comprises a plate configured to fit a user's palate and upper (maxillary) dental arch, and an electronic circuit within, on or both within and on the plate and comprising one or more detection element configured to detect one or more of, or all of, a pressure applied by a tongue of the user, a position of the tongue within an intraoral cavity (mouth) of the user, or speech sounds produced by the user, wherein the electronic circuit is configured to wirelessly provide feedback to the user based on data from the one or more detection element.

In another aspect, a speech method comprises providing an intraoral speech device in a palate of a user, the device comprising an electronic circuit and one or more detection element, using the one or more detection element to detect tongue data that comprises tongue pressure data and/or tongue position data; and wirelessly providing feedback from the device to the user based on the tongue data. In addition, data may be stored or transmitted remotely for monitoring and/or telepractice use.

Although some of the aspects of the subject matter disclosed herein have been stated hereinabove, and which are achieved in whole or in part by the presently disclosed subject matter, other aspects will become evident as the description proceeds when taken in connection with the accompanying drawings as best described hereinbelow.

### BRIEF DESCRIPTION OF THE DRAWINGS

Features and advantages of the present subject matter will be more readily understood from the following detailed description which should be read in conjunction with the accompanying drawings that are given merely by way of explanatory and non-limiting example, and in which:

FIG. 1 is a top view of an intraoral speech device according to an embodiment of the presently disclosed subject matter;

FIG. 2 is a front perspective view of a mouthpiece to which an intraoral speech device is attached according to an embodiment of the presently disclosed subject matter;

FIG. 3 is a schematic representation of an intraoral speech device in communication with an external electronic device according to an embodiment of the presently disclosed subject matter;

FIGS. 4A and 4B are schematic plan views of arrangements of detection elements on an intraoral speech device according to an embodiment of the presently disclosed subject matter;

FIG. 5 is a schematic representation of multiple intraoral speech devices in communication with an external electronic device according to an embodiment of the presently disclosed subject matter; and

FIG. 6 is a flow chart illustrating a method for speech training using an intraoral speech device in communication with an external electronic device according to an embodiment of the presently disclosed subject matter.

### DETAILED DESCRIPTION

The subject matter herein discloses a custom-fitted intraoral device augmented by a computer or mobile application or program (i.e., mobile gaming application) that is configured to improve speech efficacy leading, in some cases, to rapid and sustained correction of speech intelligibility. The novel and innovative approach aims to design and develop an intraoral speech device and mobile gaming application for speech impediment patients, and the principles disclosed herein can be applied to help train a user's speech patterns in other contexts, such as for learning foreign languages, modifying speech accents, and for singing. The speech device can be in a form of a mouthpiece-type device and can be used for speech therapy applications as well as speech diagnostic applications.

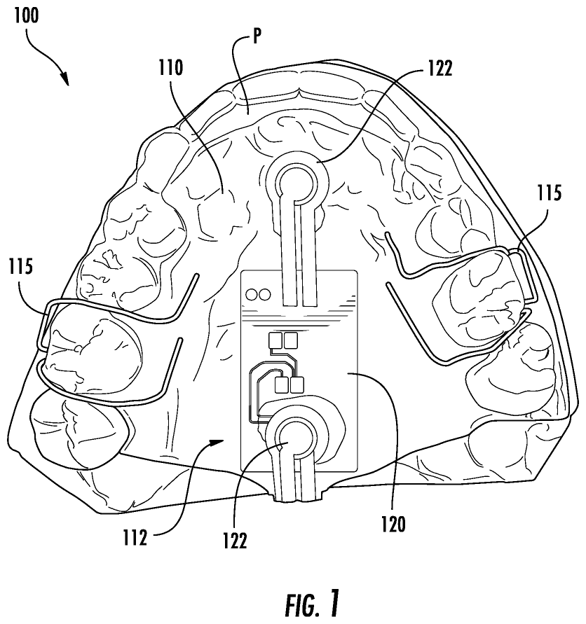

Referring to FIG. 1, an intraoral speech device, generally designated 100, comprises a customized intraoral plate 110 that is configured to conform to a user's or subject's palate P and upper dental arch. A model of the user's palate and teeth can be obtained through an impression or via 3D printing utilizing an intraoral 3D digital scan of the user's teeth, dental arches, and palate, and intraoral plate 110 can be configured to fit in the palate P and/or around the teeth and dental arch of the user's mouth. For example, intraoral plate 110 can be made from acrylic, polypropylene, or any other suitable substance that is moldable to the contours of the user's palate, and intraoral plate 110 can be flexible, inflexible, or include both portions that are flexible and portions that are inflexible. Flexibility of the plate can allow for comfort and good fit around the contours of the mouth, whereas inflexibility and flexibility of the plate can allow for detection elements to be placed with an appropriate undersurface to support tongue sensing. Intraoral plate 110 can be made of any suitable color or any color chosen by the user or professional manufacturing the plate. Alternatively, a model of the upper dental arch and palate of each user can be used to create a 3-D printed model for custom-production of the full component intraoral speech device 100.

In some embodiments, the intraoral speech device 100 can have a form that is similar to a removable orthodontic retainer. In such configurations, the device can be designed with dental wire retention clasps 115, which can be embedded in the material of intraoral plate 110 and can be suitably attached to, against, or retained on or by one or more teeth as desired. In some embodiments, for example, clasps 115 are composed of metal (e.g., stainless steel) or any other suitable material that can securely hold intraoral plate 110 in position with respect to a user's palate P and dental arches. Alternatively, referring to FIG. 2, the device can also be held in place by clear, plastic, braces-like retainer trays or materials, with or without dental clasps, such as with mouthpiece 116 which fits onto a user's teeth and holds plate 110 against a user's palate P (from FIG. 1). In either configuration, clasps 115 and/or the plate can be contoured to engage dental and tissue undercuts in the mouth. Mouthpiece 116 can be used with or without clasps 115.

Referring to FIGS. 1 and 2, in some embodiments, intraoral plate 110 can be thicker in some areas versus others. For example and without limitation, in some embodiments, intraoral plate 110 is thicker in a posterior region 112 of intraoral plate 110 (i.e., closer to the throat) compared to an anterior area of intraoral plate 110 (i.e., closer to the opening of the mouth). In such an embodiment, the thicker portion is configured to encourage the tongue to move forward, toward the opening of the mouth. In other words, the material of intraoral plate 110 in a region of the posterior palate can be thickened as such for example to prevent the dorsum of the tongue lifting with specific speech articulation errors. In addition, this thickness can further be advantageous to accommodate one or more electrical components as discussed below.

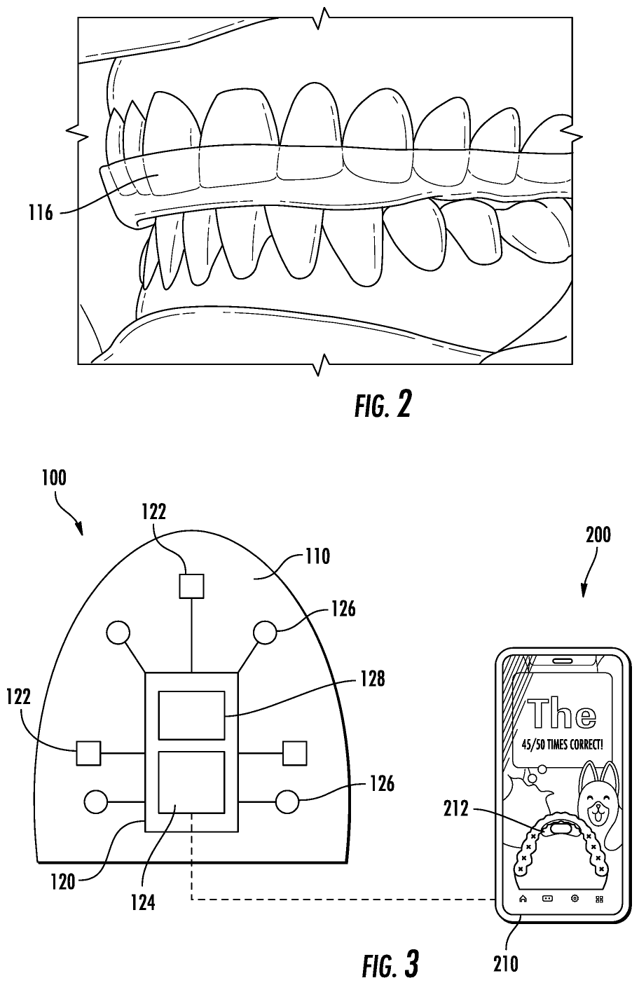

Referring to FIG. 3, in some embodiments, intraoral speech device 100 includes one or more electronic circuit(s) 120 entirely or partially embedded in intraoral plate 110 or positioned on a surface of intraoral plate 110. For example and without limitation, in some embodiments, electronic circuit 120 is a printed circuit board that is configured to collect information regarding the tongue position, placement and/or pressure of the user, and/or speech sounds. The printed circuit board may be of any shape and size, and electronic components may be placed in any location on the printed circuit board. In some embodiments, electronic circuit 120 is integrated into any desired location in the intraoral plate 110 by forming a recess (e.g., by laser-cutting) in an outer-surface of the material of intraoral plate 110 to a depth that substantially corresponds to a thickness of electronic circuit 120, and electronic circuit 120 can be embedded and retained in the recess. Detection elements within the electronic circuit may be embedded partly or wholly in any position within the plate or be positioned in any location on the surface of the plate. Regardless of the particular position, electronic circuit 120 can be formed of and/or encapsulated by a material that is sufficiently bio-compatible, waterproof, and sterilizable so as to remain protected from saliva and the intraoral environment while also not causing any irritation or discomfort to the user. In some embodiments, for example, electronic circuit 120 can be encased in a variable surface silicon, stippled waterproof silicon, or other medical-grade waterproof casing that can be embedded into the acrylic, polypropylene, or other material of intraoral plate 110.

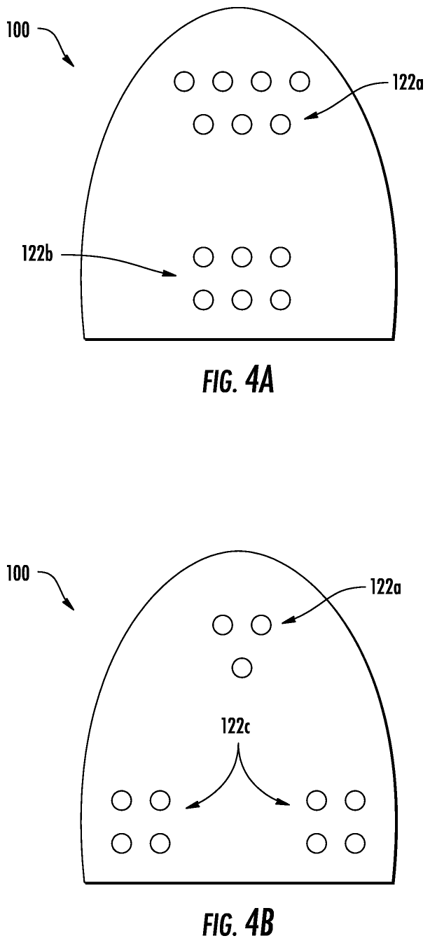

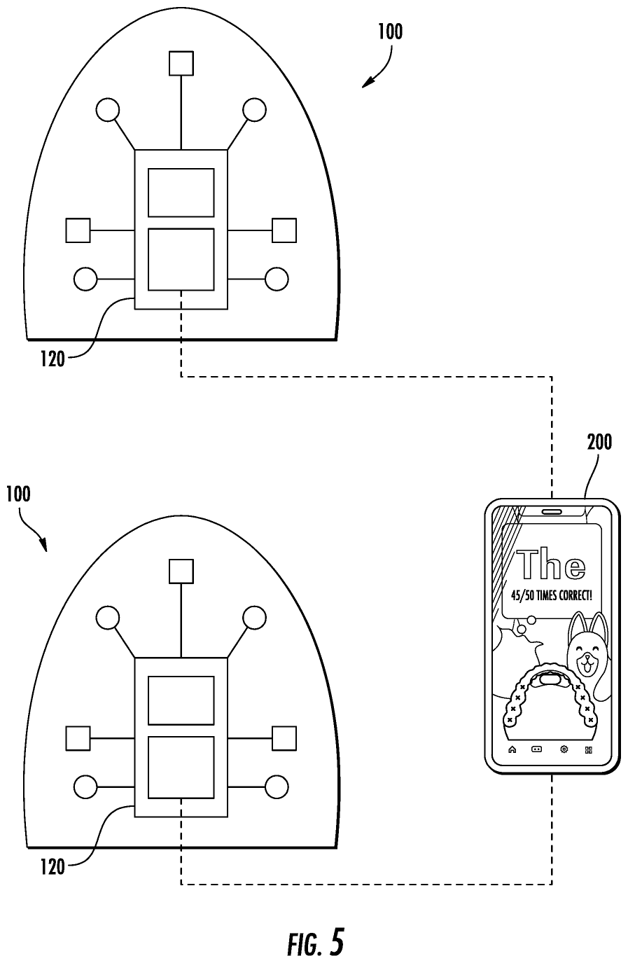

In some embodiments, one or more detection element 122 is likewise at least partially (or entirely) embedded on or within intraoral plate 110, or alternatively positioned on the surface of intraoral plate 110. Each detection element 122 can be placed in any position within the plate to identify tongue position and pressure, and/or speech sounds. The number of elements can vary from one to multiple in the one plate spaced at any distance desired. For example, three detection elements may be placed at the front of the plate at 3 mm separation and another three detection elements may be placed at the back-end of the plate for identifying correct and incorrect tongue position and pressure when making certain speech sounds (e.g. /l/, /t/, /d/, /r/). Referring to FIG. 4A, for example, the one or more detection element 122 can be arranged to be able to discern between activation of a forward set of elements 122a that are associated with correct positioning of the tongue when making speech sounds /l/, /t/, and /d/ and activation of a rearward set of elements 122b that are commonly associated with incorrect positioning of the tongue when making those sounds. Similarly, FIG. 4B illustrates that the one or more detection element 122 can likewise include an arrangement that allows for differentiation between activation of the forward set of elements 122a that are associated with a common incorrect positioning of the tongue when making speech sound /r/ and activation of a lateral set of elements 122c that are associated with a correct positioning of the tongue for that sound.

In addition, in some embodiments, the one or more detection element 122 can include ultrasonic sensors placed along the side of intraoral plate 110 for sensing tongue position at the same time. In another example, the one or more detection element 122 can be positioned around the periphery of the dental arch to detect tongue position and pressure when making sounds, such as /z/. Each detection element 122 can be one of an electrode or sensor that is positioned to sense correct and incorrect tongue positioning, pressure, or sound during speech articulation. In some embodiments, the detection element 122 can comprise one or more force sensor or strain gauge capable of low power consumption and wireless readout, and any of a variety of other suitable sensors (e.g. capacitive Micro-Electro-Mechanical-Systems (MEMS)) can be used. In some embodiments, the one or more detection element 122 can comprise an ultrasonic element comprising a transmitter and receiver. In some embodiments, the one or more detection element 122 is coated or encapsulated with a chemically and biologically inert material to ensure waterproofing within the intraoral environment.

Regardless of the particular form of the one or more detection element 122, in some embodiments the one or more detection element 122 is configured to detect one or more of a pressure applied by a tongue of the user, a position of the tongue within a mouth of the user, or speech sounds produced by the user. It is also envisioned that a plurality of detection elements 122 can be used on the same plate where the detection elements 122 are configured to detect different features. For example, one or more of the detection elements 122 can be configured to detect the pressure applied by a tongue of the user, while one or more others of the detection elements 122 is or are configured to detect the position of the tongue within a mouth of the user, and/or to detect speech sounds produced by the user.

The one or more detection element 122 can detect tongue pressure, for example, in a range of between, and including, about 0.001 kPa and 50 kPa. In some embodiments and without limitation, the one or more detection element 122 can be configured to detect and record (for example for subsequent analysis) a tongue position of the user up to within a 0.1 mm-6.0 mm resolution. In some embodiments, however, power consumption and communications system use can be minimized in configurations in which each detection element 122 is configured to supply a signal to electronic circuit 120 only where the detected pressure exceeds a minimum detection threshold. In this way, the one or more detection element 122 can be configured to communicate with the control and feedback systems discussed below when the tongue is sensed in the correct place and pressure in the mouth for the desired speech sounds. The detection elements can also be configured to provide information on incorrect tongue placement and pressure when making speech sounds. Intraoral speech device 100 can be configured such that the detected tongue pressure and/or position data are recorded for diagnostic, therapeutic, and/or research purposes. Alternatively or in addition, intraoral speech device 100 can be configured to provide real-time feedback such that data need not necessarily be recorded.

The one or more detection element 122 can be individually in communication with electronic circuit 120 to discretely detect tongue pressure at various locations on intraoral plate 110 and/or the one or more detection element 122 can be configured to operate as an array of detection elements 122 to communicate a coordinated combination of locations at which tongue pressure is detected. In some embodiments, for example, the one or more detection element 122 can be placed around intraoral plate 110 at a location or locations that substantially correspond with positions at which the tongue needs to be for specific speech sounds. For example, in some embodiments, one or more of detection element 122 is positioned to detect the tongue as being in a normal position at rest, whereas others of the one or more of detection element 122 are positioned around the palate to detect where the tongue should move to make certain sounds. In some embodiments, the one or more of detection element 122 can provide the tongue data to electronic circuit 120.

In some embodiments, the plate comprises one or more processor or microprocessor 124 configured to wirelessly provide the feedback to the user based on readings from the one or more detection element(s) 122. In some embodiments, the one or more processor or microprocessor 124 can comprise one or more integrated circuits for data processing and communication protocols. In some embodiments, electronic circuit 120 and the one or more processor or microprocessor 124 can be combined into a single chip, or they can be separate circuit elements connected through intraoral plate 110. In some embodiments, the one or more processor or microprocessor 124 is in communication with the one or more detection element 122 and electronic circuit 120 and configured to process inputs from the one or more detection element 122 and provide electrical signals to electronic circuit 120 detailing what feedback to give the user. For example and without limitation, when the one or more detection element 122 detects tongue pressure in various locations throughout the palate while the user is speaking, the one or more processor or microprocessor 124 is configured to determine whether the tongue is moving in a desired or correct position and pressure and/or force and send signals to electronic circuit 120 detailing whether the tongue position is desired or correct while the user is saying the particular words.

In some embodiments, intraoral speech device 100 further comprises a power source 128 configured for charging the various electronic components of the device. In some embodiments, for example, power source 128 can include a rechargeable battery (e.g., a lithium polymer battery (LiPo) battery) and wireless charging circuitry and/or a wired connection for charging the battery. The particular configuration for power source 128 can be selected based on suitable charge capacity, size, and safety. In some embodiments, wired charging of the battery will take place through ports in the plate that are covered by a waterproof, inert material when not being used. Wireless charging circuitry will be designed based on battery type as well as on available real estate on electronic circuit 120 for the receiver coil and regulating/charging circuitry. The charge rate profile can correspondingly be tailored to the specific battery type. In some embodiments, transduction of power from the transmitter to intraoral speech device 100 is optimized based on coil size and frequency of operation. A lines-powered wireless charging station corresponding to power source 128 can thereby permit easy charging of intraoral speech device 100 by the user. The wireless charging circuitry will be embedded full or partially within the plate and placed in any position in the plate to facilitate effective induction of the receiver coil by the transmitter coil.

All components, including the one or more detection element 122, the one or more processor or microprocessor 124, and power source 128, can be placed at any of a variety of locations on or in intraoral plate 110 depending on the user's palatal and dental anatomy and desired speech requirements.

In some embodiments, intraoral speech device 100 is configured to wirelessly provide feedback to the user regarding the tongue position, placement and/or pressure based on readings from the one or more detection element 122 while the user is talking, the feedback being configured to encourage, enable, and facilitate desired speech by the user. In some embodiments, for example, one or more onboard feedback element 126 is positioned on or at least partially or entirely embedded within intraoral plate 110. Each onboard feedback element 126 is configured to provide a physical feedback cue on correct and incorrect tongue position and pressure to the user at one or more position on intraoral plate 110. For example and without limitation, each onboard feedback element 126 can provide a vibration, electric shock, pulse, or other haptic feedback, electro-tactile feedback, ultrasound tactile feedback, thermal feedback, force feedback, and/or any of a variety of other suitable type of feedback, to the tongue or other part of the mouth to let the user know when their tongue is placed in a predetermined, correct position or to indicate that the tongue is placed in an incorrect position for the particular word, phrase, or sentence being spoken. In some embodiments, the electronic circuit further comprises devices that are configured to provide haptic feedback.

In some embodiments, for example and without limitation, electronic circuit 120 and/or the one or more onboard feedback element 126 can comprise a piezo actuator with haptic feedback or any other suitable device capable of providing haptic feedback or any other physical feedback. The feedback given by electronic circuit 120 is configured to encourage and facilitate desired speech by the user. Regardless of the particular form of physical feedback, the feedback provided inside the mouth as the user is speaking can be controlled by logic circuits contained on electronic circuit 120. Thus, in some embodiments, intraoral speech device 100 is configured to operate on its own to change or correct the speech of the user without any communication with devices outside the mouth of the user. In other words, in some embodiments, intraoral speech device 100 can, in some embodiments, operate to change or correct the speech of the user, without transmitting data to or receiving data from outside the mouth.

Alternatively or in addition, in some embodiments, electronic circuit 120 is configured to wirelessly communicate with a remote computer or other external electronic device 200, such by an app running on a smartphone or other handheld electronic device. In some embodiments, for example, electronic circuit 120 can comprise and utilize wireless communication components such as, for example and without limitation, Bluetooth components and/or Wi-Fi components. In any form, electronic device 200 can be configured to initiate feedback that can encourage tongue placement and pressure.

Alternatively or in addition to the one or more onboard feedback element 126, in some embodiments, the feedback given can comprise audio-visual feedback provided by electronic device 200, such as on a mobile device, screen, or monitor. For example and without limitation, a visual rendering 212 of the user's mouth can be displayed for the user on a mobile device, screen, or monitor of electronic device 200. The audio-visual feedback can be provided as a visual rendering 212 of the user's mouth, including indicating on visual rendering 212 where the user's tongue should be placed, where the tongue is currently being placed, and where it should be moved to with or without the appropriate tongue pressure, or any other suitable information. Such visual feedback can also be accompanied by audio cues.

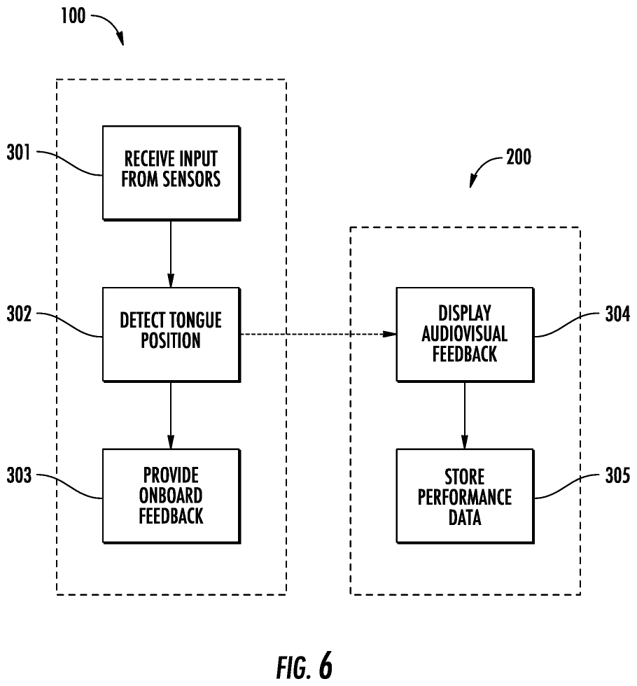

Referring to FIGS. 3 and 5, in some embodiments wireless communication components within intraoral speech device 100 are configured to communicate with one or more processing systems external to the intraoral speech device of the present disclosure. For example and without limitation, the wireless communication components can be configured to wirelessly communicate with an external electronic device 200 operating a computer or mobile application 210. To provide actionable feedback for the subject, intraoral speech device 100 can be configured to communicate with electronic device 200 with a sufficiently short response time that the audio-visual feedback provided by computer or mobile application 210 is effectively instantaneous. There is evidence that human visual reaction time is between about 180-330 milliseconds, and thus providing a sampling rate within or around this range can be perceived as instant. In some embodiments, for example, a response time of about 5 □s or less can be considered sufficiently rapid so as to provide real-time feedback without significant delay.

In some embodiments, computer or mobile application 210 can comprise a mobile application game, a mobile application, a game operating on a cloud server, or any other suitable computer or mobile application. In some embodiments, computer or mobile application 210 can comprise a mobile game application that is configured to interact with intraoral speech device 110 and give the user an interface to track progress, play games that help the user improve their speech, play games that allow them to compete with other users, or other suitable gaming platform. Such apps can, for example and without limitation, include games displayed and played on electronic device 200 that electronically communicates with intraoral speech device 100. Such games can for example include interactive activities and/or exercises encouraging and even rewarding a wearer of the speech device to engage in proper, desired, and/or correct tongue positioning and placement with correct pressure applied by the tongue for desired speech. The mobile app can also be configured to provide feedback on speech sounds while the speech sounds are being made and recorded such as for further, later analysis. The apps or games can, for example and without limitation, comprise the user (wearer) of intraoral speech device 100 creating a character in the app or game.

As described herein, in some embodiments, the external processing system operating the computer or mobile application 210 can be in communication with one or more intraoral speech devices 100. For example, as shown in FIG. 3, the external electronic device 200 could be a mobile phone (or tablet, computer, etc.) connected wirelessly to a single intraoral device 100. In some other embodiments, for example, the illustration in FIG. 5, a single external electronic device 200 can be used to communicated wirelessly with two or more intraoral speech devices 100. In this way, for example a server or other computer system can remotely communicate with multiple devices, potentially in the same room, such as in a classroom. In some embodiments, in either the configuration illustrated in FIG. 3 or FIG. 5, external electronic device 200 can be configured to transmit data to or receive data from intraoral speech device 100.

In some embodiments, the computer or mobile application 210 is configured to wirelessly record and/or display data to the user (or any other person), including desired or correct tongue position, placement and/or pressure, and the correct speech sounds. In some embodiments, the computer or mobile application 210 is configured to record data (i.e., tongue position and pressure data, desired or correct tongue position, etc.) from the intraoral speech device. The computer or mobile application 210, in some embodiments, can be configured to wirelessly transmit and receive data from another computer, server, or computing system. For example and without limitation, the computer or mobile application 210 can be configured to wirelessly transmit or display helpful feedback to the user via an application on their phone, email or other means. Such feedback could include accuracy percentage, details on how to get better, high scores on particular games played, or any other suitable data. The app could also play back the sound made and related to tongue positioning provide a score on success. In some embodiments, the gamification features of the app and ability to be a part of a virtual community will provide motivational support to regular intraoral device supporting correct tongue positioning and pressure for production of the desired speech sounds. Known as the Hawthorne effect, recording of data and remote monitoring also support regular device use with associated achievement of desired speech.

In some embodiments, the computer or mobile application 210, whether it be a mobile application, mobile game, or gaming application, can be downloaded by a speech-language pathologist or other health professional with the user and the health professional can help register or input user data and prescribe various exercises to be performed with intraoral speech device 100. In some embodiments, the computer or mobile application 210 can be configured to give awards or rewards to the user in return for good compliance with daily exercises. This can further increase the success of the device. In some embodiments, the computer or mobile application 210 is configured such that the user can create an avatar and compete with others for compliance. In some embodiments, the computer or mobile application 210 is configured to register desired or correct tongue positioning and pressure and use this information to either change or correct bad placement or reward good placement and to inform the user about what steps to take to improve their speech.

In some further embodiments, the computer or mobile application 210 is configured to display daily, weekly, and/or monthly data about the tongue placement and pressure, making of speech sounds, and speech exercises allowing the speech-language pathologist and the user to see their progress. These data may be stored to aid system iterations and for remote monitoring of progress. In some embodiments, any games or exercises displayed or played on the computer or mobile application 210 are configured to improve the speech of the user. In some embodiments, the computer or mobile application 210 is configured to transmit data wirelessly or via a wired connection, from electronic device 200 on which it is operating to the speech-language pathologist or other health professional, regarding the user's engagement and results from the exercises and games as well as the user's progress, if any, for diagnostic, therapeutic, and/or research purposes. Additionally, in some embodiments, the computer or mobile application 210 is configured such that a telehealth or video chat feature can be performed (i.e., between the user and the speech-language pathologist or any other appropriate person).

In another aspect, the subject matter of the disclosure herein includes a speech system comprising one or more intraoral speech devices 100 as described herein and one or more electronic devices 200 as described herein, each electronic device 200 being configured to operate a computer or mobile application 210 in communication with one or more of the one or more intraoral speech devices 100.

In a further aspect, the subject matter of the disclosure herein includes a speech training method as illustrated in FIG. 6. Intraoral speech device 100 in accordance with one of the above embodiments is provided in a palate and upper dental arch of a user, intraoral speech device 100 including at least an electronic circuit 120 and one or more of detection element 122. In a sensing step 301, an input is received from the one or more of detection element 122. From this input, a tongue position and pressure of the user is detected by identifying the tongue position that most closely corresponds to the combination of inputs received from the one or more of detection element 122 in a tongue detection step 302. In some embodiments, the detected tongue position is compared to a desired tongue position and pressure, and an onboard feedback step 303 can include providing physical feedback to the user on intraoral speech device 100. As discussed above, such onboard feedback can include vibration, electric shock, pulse, or other haptic feedback, electro-tactile feedback, ultrasound tactile feedback, thermal feedback, force feedback, and/or any of a variety of other suitable type of feedback.

Alternatively or in addition, in some embodiments, the identification of the tongue position, placement and/or pressure is communicated to an external electronic device 200, and an audio-visual feedback is provided to the user based on the evaluation of the correctness of the tongue position and pressure for a given speech pattern in a display step 304. As discussed above, in some embodiments, tongue position and pressure data, desired or correct tongue position, or other relevant data from intraoral speech device 100 can be stored on the electronic device 200 or communicated to another computer, server, or computing system in a data collection step 305. As discussed above, such data storage can be used to track usage of intraoral speech device 100 and document progress of the user for diagnostic, therapeutic, and/or research purposes.

The present subject matter can be embodied in other forms without departure from the spirit and essential characteristics thereof. The embodiments described therefore are to be considered in all respects as illustrative and not restrictive. Although the present subject matter has been described in terms of certain specific embodiments, other embodiments that are apparent to those of ordinary skill in the art are also within the scope of the present subject matter.

1. An intraoral speech training system comprising:
an intraoral plate configured to fit a palate in a mouth of a user; and
an electronic circuit within the plate and comprising one or more detection element configured to detect one or more of a pressure applied by a tongue of the user, a position of the tongue within a mouth of the user, or speech sounds produced by the user;
wherein the electronic circuit is configured to wirelessly provide feedback to the user based on data from the one or more detection element to indicate tongue position, placement or pressure on the one or more detection element.

2. The intraoral speech training system of claim 1, wherein the plate is made from acrylic or polypropylene material, or another material.

3. The intraoral speech training system of claim 1, wherein the plate comprises contours and surface modifications that are configured to encourage the tongue to be positioned in a desired or correct position corresponding to the desired speech.

4. The intraoral speech training system of claim 1, wherein the feedback comprises one or more of haptic or tactile feedback.

5. The intraoral speech training system of claim 1, wherein the provided feedback is audio-visual feedback displayed on a mobile device, screen, or monitor.

6. The intraoral speech training system of claim 1, comprising one or more microprocessor configured to provide the feedback based on the readings or data from the one or more detection element.

7. The intraoral speech training system of claim 1, wherein the plate further comprises a rechargeable battery within the plate and configured to provide power to the electronic circuit.

8. The intraoral speech training system of claim 1, wherein the plate is retained on teeth of the user using clasps and engagement of tissue and dental undercuts.

9. The intraoral speech training system of claim 1, wherein the plate further comprises a wireless communication component.

10. The intraoral speech training system of claim 1, wherein the electronic circuit is encased in a stippled waterproof silicon or other suitable medical-grade material casing embedded into the acrylic or polypropylene or other suitable material of the plate.

11. The intraoral speech training system of claim 1, wherein the one or more detection element comprise a plurality of detection elements configured as an array of sensors or individual sensors positioned on or in the plate.

12. The intraoral speech training system of claim 1, wherein the plurality of detection elements comprise one or more detection elements configured to detect the pressure applied by the tongue of the user, while one or more others of the plurality of detection elements is configured to detect the position of the tongue within the mouth of the user and/or to detect speech sounds produced by the user.

13. The intraoral speech training system of claim 1, wherein the feedback comprises vibrations, electric shocks, or pulses.

14. The intraoral speech training system of claim 1, comprising a mobile device configured to operate a mobile application in communication with the electronic circuit.

15. The intraoral speech training system of claim 14, wherein the mobile application is configured to communicate with the electronic circuit via wireless communication components.

16. The intraoral speech training system of claim 14, wherein the mobile application is configured to wirelessly record and display data to the user, including:
correct tongue positioning and pressure;
speech sounds as the speech sounds are being made; and
daily, weekly, monthly, or yearly progress graphs, indicating a progression the user is making.

17. The intraoral speech training system of claim 14, wherein the mobile application is a gaming application.

18. The intraoral speech training system of claim 14, wherein the mobile application is configured to transmit data remotely to one or more other computing systems.

19. A speech training method comprising:
providing an intraoral speech device in a palate and upper dental arch of a user, the device comprising an electronic circuit and one or more detection element;
using the one or more detection element to detect speech sounds or tongue data, the tongue data comprising one or more of tongue pressure data and tongue position data; and
providing feedback from the device to the user based on the detected speech sounds or tongue data.

20. The method of claim 19, wherein the device has a thicker dimension in one or more portions of the palate that are configured to encourage desired tongue positioning.

21. The method of claim 19, wherein providing feedback comprises providing one or more of haptic or tactile feedback.

22. The method of claim 19, wherein providing feedback comprises wirelessly providing audio-visual feedback displayed on a mobile device, screen, or display.

23. The method of claim 19, comprising retaining the intraoral speech device on teeth of the user using clasps and engagement of tissue and dental undercuts.

24. The method of claim 19, wherein the intraoral speech device comprises transmitting the tongue data using wireless communication components.

25. The method of claim 19, wherein the one or more detection element comprise a plurality of detection elements configured as an array of sensors or individual sensors positioned in or on the plate;
wherein using the one or more detection element comprises detecting speech sounds and/or tongue data using the array of sensors or individual sensors.

26. The method of claim 25, comprising using one or more of the detection elements to detect speech sounds, and using one or more others of the detection elements to detect the position of the tongue within the mouth of the user and/or to detect speech sounds produced by the user.

27. A computer program product configured to operate on an intraoral speech therapy device placed in a palate of a user, the intraoral speech therapy device comprising an electronic circuit and one or more detection element, the computer program product configured to implement a method comprising:
using the sensors to detect tongue data that comprises one or more of tongue pressure data or tongue position data;
recording of speech sounds made; and
providing feedback from the device to the user based on the tongue and speech sound data.
